(19) United States
(12) Patent Application Publication
(10) Pub. No.: US 2005/0019918 A1
(43) Pub. Date: Jan. 27, 2005

(54) Treatment of cancer by inhibiting BRAF expression

(76) Inventors: Hidetoshi Sumimoto, Tokyo (JP); Yutaka Kawakami, Tokyo (JP); Makoto Miyagishi, Tokyo (JP); Kazunari Taira, Tokyo (JP)

Correspondence Address: MORGAN & FINNEGAN, L.L.P., 3 WORLD FINANCIAL CENTER, NEW YORK, NY, 10281-2101, US

(21) Appl. No.: 10/834,665

(22) Filed: Apr. 29, 2004

(30) Foreign Application Priority Data
Jun. 3, 2003 (JP) 2003-158634

(51) Int. Cl. C07H 21/02; A61K 48/00

(52) U.S. Cl. 435/375; 514/44; 536/23.1

(57) ABSTRACT

The present invention relates to a therapeutic method using RNAi directed at BRAF, of which the point mutation, especially V599E, occurs frequently in melanomas. RNAi specific for the mutated BRAF will provide a specific therapeutic intervention for cancers such as malignant melanoma. Several target sequences for RNAi were selected in the protein coding region of the BRAF mRNA. The short hairpin RNA expression cassette was constructed on the lentiviral vector. One recombinant viral vector for the mutated BRAF V599E and two other vectors sites for wild type BRAF were constructed to infect various malignant melanoma cell lines, and the effects on the growth inhibition and the signaling of MAPK pathway were examined. The inhibitory effect on the invasion ability of malignant melanoma cell line and in vivo growth of a malignant melanoma cell line were examined.

### TECHNICAL FIELD

The present invention involves the use of RNAi and relates to a double-stranded RNA that can inhibit the expression of a mutated BRAF (V599E) gene and the like (siRNA: small interfering RNA), a double-stranded RNA expression cassette that can express a double-stranded RNA, a double-stranded RNA expression vector containing a double-stranded RNA expression cassette; a preventive/therapeutic agent of cancer such as malignant melanoma and the like having these as active ingredient; and a method for preventing/treating cancer such as malignant melanoma and the like by administering these, or the like.

### BACKGROUND ART

During the generation process of living organisms, cells differentiate to cells having various characters, while their growth, life and death are stringently controlled. Moreover, as for adults after generation, the growth, differentiation and death of each cell are stringently controlled to maintain the constancy as an individual. In other words, the destiny of each cell is controlled by that the extracellular signals such as hormone, neutrotansmitter, cell growth factor, cytokine and the like, transmit accurately into the cells, mediated by the receptor on the cell membrane. The mechanism of transmitting the extracellular signal to the nucleus of the cells and controlling the genetic information is called the intracellular signaling mechanism, and the consecutive reaction of intracellular protein interaction of this mechanism is called the intracellular signaling pathway. The intracellular signaling pathway transmit the signal by repeating the process of receiving the upstream signal to become active, and transmitting the signal downstream to become inactive.

The mitogen-activated protein kinase (MAPK) pathway which is one of intracellular signal transduction system plays an important role in the growth/differentiation signal of cells. MAPK is a protein phosphorylated enzyme, which the molecular weight is approximately 40 thousands and forms a phosphorylated cascade that is MAP kinase kinase kinase (MAPKKK)→MAP kinase kinase (MAPKK)→MAP kinase (MAPK), in various cell types of eukaryote. This cascade becomes active in the downstream of protooncogene ras and induces cell differentiation, arrest of cell growth or enhancement of cell motility, not only working as cell growth signal. Furthermore, as constitutive hyperfunction of MAPK is observed in many cancer cells, thus its specific inhibition is related with antitumor therapy.

As for MAPK pathway, point mutation of BRAF which is also one of MPKKK in malignant melanoma is detected with a high frequency (66%), and the association with the malignant transformation is suggested. All the mutations are observed in the activated region of kinase domain or in the adjacent region, and it has been reported by the analysis of some of the mutation appearing frequently (V599E, L596E, G463V, G468A), that kinase activity of BRAF is increased by mutation and that as a result, ERK is activated and moreover, that the mutated BRAF has the transformation ability of NIH3T3 cells (WO99/32619). Furthermore, in many malignant melanoma and malignant melanoma tissues, the enhancement of MAPK activity has been reported constantly (WO01/36646). These reports suggest that mutated BRAF is an oncogene being highly associated with the development of malignant melanoma, and that can be a molecular target for treatment of malignant melanoma. However, in the assay system above mentioned, the influence of the excessive amount of mutated BRAF having far more exceeded the physiological expressing level on MAPK or the association with the malignant transformation remains unclear.

On the other hand, it has been found in a certain living organism (Caenorhabditis elegans), that a double-stranded RNA (dsRNA) can inhibit specifically gene expression (WO99/32619; Nature, 391, 806-811, 1998). This phenomenon is called RNAi (RNA interference), being a phenomenon wherein dsRNA consisting of sense RNA and antisense RNA homologous to a certain gene disrupts the homologous part of the transcript of that gene (mRNA). This phenomenon has been further observed in different animals (Cell, 95, 1017-1026, 1998; Proc. Natl. Acad. Sci. USA, 95, 14687-14692, 1998; Proc. Natl. Acad. Sci. USA, 96, 5049-5054, 1999) or in lower eucaryotic cells including plants (Proc. Natl. Acad. Sci. USA, 95, 13959-13964, 1998).

At the time RNAi has been found, in mammalian cells, when dsRNA larger than 30 base pairs were introduced into cells, as a non-specific gene silencing occurs by the induction of interferon response and the specific gene expression inhibition by RNAi is no longer observed, it was believed that the use in mammalian cells was difficult. However, in 2000, it was suggested that RNAi can occur even in early mouse embryo or mammalian cultured cells, and thus it has been clear that the inducing mechanism of RNAi is also present in mammalian cells (WO01/36646; FEBS Lett, 479, 79-82, 2000).

It is clear that it is useful if the expression of a particular gene or gene cluster can be inhibited also in mammals by using such function of RNAi. Many diseases (cancer, endocrinopathy, immune diseases and the like) are developed in mammals when some particular gene or gene cluster is expressed abnormally, and the inhibition of the gene or gene cluster can be used for the treating these diseases. Moreover, it happens that diseases develop due to the expression of mutated protein, and in these cases, by inhibiting the expression of mutated allele, the treatment of the disease could be possible. Moreover, it is said that such gene-specific inhibition can also be used to treat for example viral diseases caused by retrovirus such as HIV (viral genes in retrovirus are expressed by being integrated in the genome of these hosts.).

As for dsRNA inducing RNAi function, it was first believed that it was necessary to introduce dsRNA larger than 30 bp into cells, but recently it has been clear that dsRNA being shorter (21-23 bp) (siRNA: small interfering RNA) can induce RNAi without showing cytotoxicity even in mammalian cell lines (Nature, 411, 494-498, 2001). SiRNA is recognized to be a powerful tool to inhibit gene expression in all of the development stages of somatic cells, and it can be used in progressive genetic diseases and the like as a method to inhibit gene expression being the cause of the disease, before pathogenesis.

As for the frequency of the mutation, it is detected in a wide range in various cancers including malignant melanoma, and particularly, the frequency of mutation is significantly high in malignant melanoma (66%) (Nature, 417, 949-954, 2002). Moreover, the mutation is concentrated in kinase domain and the point mutation of V599E (point mutation wherein the 599th valine (V) is changed to glutamic acid (E)) constitutes 80%. These mutated BRAF is accompanied by the increase of kinase activity and transformation activity of NIH3T3 cells, the association with the development of malignant tumor is strongly suggested. Moreover, in many malignant melanoma cell lines or malignant melanoma tissues, the constitutive enhancement of MAPK activity is observed (Cancer Reserch, 63, 756-759, 2003), and the association with the malignant transformation is suggested. By the examination of the present inventors, constitutive enhancement of MAPK activity was observed in many malignant melanoma cell lines, but its level is various and the association with the presence of V599E mutation is not confirmed. This suggests the possibility that mutated BRAF on physiological level is not a factor to determine MAPK activity by itself. However, there is no report on the therapeutic system having mutated and non-mutated BRAF as target until now.

The object of the present invention is to prepare by using RNAi method, a double stranded RNA (siRNA) that can inhibit the expression of BRAF gene such as mutated BRAF (V599E) gene, a double-stranded RNA expression cassette that can express double-stranded RNA, and a double-stranded RNA expression vector containing the double-stranded RNA expression cassette, and to provide a high-security therapeutic method specific to a cancer such as malignant melanoma and the like using BRAF that can induce RNAi cancer-specifically and has hypermutaion, as a molecular target.

### DISCLOSURE OF THE INVENTION

The present inventors have improved siRNA expressing system (Nature Biotechnology, 19, 497-500, 2002) by plasmid vectors using U6 promoter that has been developed by the present inventors, and have developed lentiviral vector system in which siRNA expression is maintained in a stable manner and transgenic efficiency is higher. By using a lentiviral vector system wherein the siRNA expression is maintained in a stable manner, the present inventors have constructed siRNA expression vector specific for V599E mutation targeting V599E mutation sites and siRNA expression vector non-specific for mutation. In other words, by setting several sites for the target sequence of RNAi in coding region of BRAF mRNA, and by constructing the expressing form of siRNA, which is dsRNA and that shows RNAi effect on the lentiviral vectors and a recombinant viral vector was constructed by setting these target sequences, one is the site containing V599E and the other two are the sites containing no V599E.

Next, to investigate BRAF RNAi effect in vitro, various malignant melanoma cell lines wherein BRAF V599E mutation is positive or negative, were infected with recombinant viral vectors above-mentioned, and then siRNA expression vector wherein the effect was observed by Western Blot Analysis was selected. Next, after various malignant melanoma cell lines with or without BRAF V599E mutation were infected with these siRNA expressing viral vectors, their RNAi effects on the proliferation and MAPK activity was investigated and their correlation was studied. As a result, the inhibitory effect of siRNA specific for BRAF V599E mutation was confirmed with siRNA expression vector specific for BRAF V599E mutation. Furthermore, specific inhibition of BRAF expression by RNA interference inhibited strongly the signaling of MAPK pathway, and induces a strong proliferation inhibition or cell death. Moreover, by infecting A375 mel cells with siRNA expression lentiviral vector targeting BRAF, a significant inhibition of invasion ability was observed by matrigel invasion assay. Furthermore, it was found that the growth of the subcutaneously implanted tumor with A375 me1 cells, which had been infected with siRNA lentiviral vectors, was inhibited in vivo. Thus, the present invention has been completed according to this knowledge.

In other words, the present invention relates to a double-stranded RNA that can inhibit BRAF gene expression, being consisted of a sense strand RNA and an anti-sense strand RNA homologous to a particular sequence being the target of BRAF mRNA (“1”); the double-stranded RNA according to “1”, wherein the BRAF gene is a mutated BRAF gene (“2”); the double-stranded RNA according to “2”, wherein the mutated BRAF gene is a mutated BRAF (V599E) gene (“3”); the double-stranded RNA according to any of “1” to “3”, wherein the particular sequence being the target of BRAF mRNA is a target sequence containing the mutation site of mutated BRAF mRNA (“4”); the double-stranded RNA according to “4”, wherein the target sequence containing the mutation site of mutated BRAF mRNA consists of the RNA derived from the base sequence shown in Seq. ID No.2 and its complementary sequence (“5”); the double-stranded RNA according to any of “1” to “3”, wherein the particular sequence being the target of BRAF mRNA comprises the RNA derived from the base sequence shown in Seq. ID No. 3 or 4 and its complementary sequence (“6”); and the double-stranded RNA according to any of “1” to “6”, wherein the particular sequence being the target of BRAF mRNA is the base sequence of 19-21 bp (“7”).

Furthermore, the present invention relates to a double-stranded RNA expression cassette that can express the double-stranded RNA according to any of “1” to “7”, being consisted of a sense strand DNA-linker-anti-sense strand DNA of the particular sequence of BRAF gene (“8”); the double-stranded RNA expression cassette according to “8”, being consisted of the base sequence shown in Seq. ID No. 5 (“9”); the double-stranded RNA expression cassette according to “8”, being consisted of the base sequence shown in Seq. ID No. 6 or 7 (“10”); a double-stranded RNA expression vector, wherein the double-stranded RNA expression cassette according to any of “8” to “10” is connected to the downstream of the promoter (“11”); the double-stranded RNA expression vector according to “11”, being a HIV lentiviral vector (“12”); an inhibitor of BRAF gene expression, having as active ingredient the double-stranded RNA according to any of “1” to “7”, the double-stranded RNA expression cassette according to any of “8” to “10”, or the double-stranded RNA expression vector according to “11” or “12” (“13”); a preventive and/or therapeutic agent of cancer, having as active ingredient the double-stranded RNA according to any of “1” to “7”, the double-stranded RNA expression cassette according to any of “8” to “10”, or the double-stranded RNA expression vector according to “11” or “12” (“14”); the preventive and/or therapeutic agent of cancer according to “14”, wherein the cancer is caused by the mutation or enhancement of expression of BRAF gene (“15”); and the preventive and/or therapeutic agent of cancer according to “14”, wherein the cancer is a malignant melanoma (“16”).

Moreover, the present invention relates to a method for inhibiting BRAF gene expression, wherein the double-stranded RNA according to any of “1” to “7”, the double-stranded RNA expression cassette according to any of “8” to “10”, or the double-stranded RNA expression vector according to “11” or “12” is introduced in vivo, into tissues or cells (“17”); a method for preventing and/or treating cancer, wherein the double-stranded RNA according to any of “1” to “7”, the double-stranded RNA expression cassette according to any of “8” to “10”, or the double-stranded RNA expression vector according to “11” or “12” is introduced in vivo, into tissues or cells (“18”); the method for preventing and/or treating cancer according to “18”, wherein the cancer is a cancer caused by the mutation or enhancement of expression of BRAF gene (“19”); the method for preventing and/or treating cancer according to “18”, wherein the cancer is a malignant melanoma (“20”).

As it is clear from the results of the Examples, it was demonstrated that the specific inhibition of BRAF expression by RNA interference inhibited strongly the signaling of MAPK pathway, and it induces a strong growth inhibitory effect or cell death inducing effect according to the inhibition of growth signal. It is strongly suggested that the in vitro cell growth inhibitory effect induces as well the in vivo growth inhibitory effect, and it can be expected as a useful gene medicine. Moreover, as the siRNA specific for V599E mutation of the present invention acts specifically to the mutation place observed with a high frequency in malignant melanoma, an effect of specific action to cancer cells without injuring normal cells can be expected, it can be used as a molecular target therapy which is highly safe. As for the siRNA non-specific to V599E mutation, if a therapeutic window can be provided according to the difference of BRAF expression between cancer cells and normal cells, there is a possibility that a selective therapeutic effect occurs. Moreover, these siRNAs are very useful not only for the application in medical field, but also as a tool for basic research of BRAF-MAP kinase signaling pathway.

### BRIEF DESCRIPTION OF DRAWINGS

FIG. 1 is a figure that shows the structure of siRNA expression HIV lentiviral vector plasmid of the present invention.

FIG. 2 is a figure that shows the DNA base sequence corresponding to each siRNA of the present invention.

FIG. 3 is a figure that shows the result of Western Blot Analysis of BRAF after infection to 293T cells and A375 mel cells of the present invention.

FIG. 4 is a figure that shows the result of comparison of the growth in 10 types of malignant melanoma cell lines that were infected with siRNA expression vector of the present invention at 50-100 MOI, and the cells infected with GL3B expression vector for control.

FIG. 5 is a figure that shows the results of Western Blot Analysis of the protein extracted from various malignant melanoma cell lines after infection with siRNA expression vector of the present invention.

FIG. 6 is a figure that shows the results of matrigel invasion assay showing the inhibitory effect of invasion ability of malignant melanoma cell lines (a) and the results of Western Blot Analysis of matrix metalloproteinase-2 (b) and □1 integrin (c).

FIG. 7 is a figure that shows the chronological change of the tumor volume, showing the inhibitory effect of siRNAs on the growth of a malignant melanoma cell line in vivo.

### BEST MODE FOR CARRYING OUT THE INVENTION

As for the double stranded RNA of the present invention, there is no specific limitation as long as it is a double-stranded RNA that can inhibit the expression of BRAF gene and consists of a sense strand RNA and anti-sense strand RNA homologous to the particular sequence within the BRAF mRNA. Though there is no limitation for the origin of BRAF gene, a human-derived BRAF gene is preferable, and a mutated BRAF gene is most preferable. As for the mutated BRAF gene, mutated BRAF representing as V599E, L596E, G463V, G468A can be exemplified concretely, and mutated BRAF (V599E) gene (a mutated gene of BRAF gene, wherein the 1857th T is substituted to A) consisting of the base sequence shown in Seq. ID No.1, being strongly related with the development of malignant melanoma, is exemplified most preferably.

As for the particular sequence being the target of BRAF mRNA above mentioned, it relates to the part sequence of the specific region of BRAF mRNA, preferably the part sequence wherein the base sequence length is 19-21 bp, the particular sequence for BRAF mRNA is most preferable, and the target sequence containing the mutation site of mutated BRAF mRNA. As for the target sequence containing the mutation site of the mutated BRAF mRNA, a double-stranded RNA containing the mutation site of mutated BRAF (V599E) gene and consisting of RNA derived from the base sequence GCT ACA GaG AAA TCT CGA T shown in Seq. ID No. 2 in the sequence listing (19 mer of 1850-1868 of the base sequence shown in Seq. ID No. 1) and its complementary sequence can be exemplified concretely. Moreover, although it is not a particular sequence containing the mutation site of the mutated BRAF gene, as a target sequence that can inhibit BRAF mRNA expression, the double-stranded RNA consisting of RNA derived from the base sequence GCC ACA ACT GGC TAT TGT TA shown in Seq. ID No. 3 in the sequence listing (20 mer of 1624-1643 of the base sequence shown in Seq. ID No. 1) and its complementary sequence, or the double stranded RNA consisting of RNA derived from the base sequence shown in Seq. ID No. 4 in the sequence listing (21 mer of 1669-1689 of the base sequence shown in Seq. ID No. 1) and its complementary sequence can be exemplified concretely. Concerning these siRNA non-specific to V599E mutation, if a therapeutic window can be provided according to the difference of BRAF expression between cancer cells and normal cells, the possibility of a selective therapeutic effect can be expected.

Moreover, the sense-strand RNA homologous to the particular sequence that is the target of BRAF mRNA relates for example to an RNA derived from the DNA sequence shown in Seq. ID Nos. 2 to 4 above mentioned, and the anti-sense strand RNA homologous to the particular sequence that is the target of BRAF mRNA relates to the sense-strand RNA above mentioned and its complementary RNA. The double-stranded RNA of the present invention is generally constructed as siRNA wherein these sense strand RNA and anti-sense strand RNA are bound, but as a matter of convenience, a double-stranded RNA constructed as siRNA of mutated sense strand RNA sequence wherein one or several bases are deleted, substituted or added in sense strand RNA sequence with a mutated anti-sense strand RNA sequence complementary to said mutated sense strand RNA sequence is also within the scope of the present invention.

To prepare the double-stranded RNA (dsRNA) of the present invention, known methods such as a method using synthesis, a method using transgenic technology and the like can be used. With the method using synthesis, the double-stranded RNA can be synthesized by the common method according to the sequence information. Moreover, with the method using the transgenic technology, the dsRNA can be prepared by constructing an expression vector integrating a sense strand DNA or anti-sense strand DNA, introducing the vector into the host cell, and then by obtaining a sense-strand RNA or anti-sense strand RNA generated by transcription, respectively. However, it is preferable to prepare the intended double-stranded RNA with the expression and construction in vivo, by constructing a double-stranded RNA expression cassette consisting of the sense-strand DNA-linker-anti-sense strand DNA of the particular sequence of BRAF gene, and by binding the double-stranded RNA expression cassette to the downstream of the promoter of the expression vector.

As for the double-stranded RNA expression cassette of the present invention above mentioned consisting of the sense-strand DNA-linker-anti-sense strand DNA of the particular sequence of BRAF gene, the double-stranded RNA expression cassette consisting of the base sequence GCT ACA GaG AAA TCT CGA T TTCAAGAGA ATC GAG ATT TCt CTG TAG C ttttt shown in Seq. ID No.5 in the sequence listing having TTCAAGAGA as linker sequence, the double-stranded RNA expression cassette consisting of the base sequence GCC ACA ACT GGC TAT TGT TA TTCAAGAGA TA ACA ATA GCC AGT TGT GGC ttttt shown in Seq. ID No.6 in the sequence listing, the double-stranded RNA expression cassette consisting of the base sequence GTA TCA CCA TCT CCA TAT CAT TTCAAGAGA ATG ATA TGG AGA TGG TGA TAC ttttt shown in Seq. ID No. 7 in the sequence listing can be exemplified concretely. These double-stranded RNA expression cassettes can form a double-stranded RNA consisting of a sense strand RNA corresponding to the sense strand DNA and an anti-sense strand RNA corresponding to the anti-sense DNA, when it is transcribed in the host cell.

Moreover, as for the expression vector that can introduce the double-stranded RNA expression cassette in the downstream of the promoter, examples include murine leukemia retroviral vector (Microbiology and Immunology, 158, 1-23, 1992), adeno-associated viral vector (Curr. Top. Microbiol. Immunol., 158, 97-129, 1992), adenoviral vector (Science, 252, 431-434, 1991), liposomes and the like. However, HIV lentiviral vector is preferable from the point of view that it is efficient to the non-dividing cells and that long-term expression is possible. Moreover, these expression systems can include a controlling sequence that not only promotes expression but controls the expression. The introduction of the double-stranded RNA expression cassettes to these expression vector can be performed by a common method, and the double-stranded RNA expression vector of the present invention can be constructed for example by introducing the double-stranded RNA expression cassette to the downstream of an appropriate promoter of these expression vectors.

As for the inhibitor of BRAF gene expression of the present invention or the preventive and/or therapeutic agent of cancer of the present invention, there is no specific limitation as long as it has the double-stranded RNA of the present invention, the double-stranded RNA expression cassette of the present invention, or the double-stranded RNA expression vector of the present invention as active ingredient. Moreover, when administering or introducing the inhibitor of BRAF gene expression or preventive/therapeutic agent of cancer in vivo, into tissues, cells and the like of mammals, it can be used with various compounding ingredients for prescription such as carrier pharmaceutically acceptable and generally used in this field, bonding agent, stabilizing agent, diluting agent, diluent, pH buffer agent, disintegrator, solubilizer, solvent adjuvant, isotonic agent and the like. As for the pharmaceutical composition used with the carrier pharmaceutically acceptable, it can be prepared in a form that is known in the pharmaceutical field, according to the administration form, that is for example oral administration (including intraoral or sublingual administration), or parenteral administration (injection and the like) and the like.

Moreover, as for the inhibition method of BRAF gene expression of the present invention or the preventive and/or therapeutic method of cancer of the present invention, there is no specific limitation as long as it is a method to introduce the double-stranded RNA, the double-stranded RNA expression cassette or the double-stranded RNA expression vector of the present invention above-mentioned in vivo, into tissues or cells of mammals, and as for the method for introducing the double-stranded RNA, double-stranded RNA expression cassette, or double-stranded RNA expression vector in vivo, into tissues or cells of mammals, oral or parenteral administration method can be exemplified. For example, it can be administered orally in a dosage form such as powder medicine, granule, capsule, syrup, suspension and the like, or it can be administered parenterally by injection in a dosage form of solution, emulsion, suspension and the like, or it can be administered intranasally in form of spray. Moreover, the dosage can be selected appropriately according to the disease, body weight of the patient, administration form and the like.

As for the cancer that is the target of the preventive/therapeutic agent of cancer of the present invention, or the preventive/therapeutic method of cancer of the present invention, cancer caused by BRAF gene mutation or enhacement of BRAF gene expression can be exemplified, and more concretely, colon cancer, lung cancer, breast cancer, ovary cancer, brain tumor, thyroid cancer and the like can be exemplified beside malignant melanoma.

### EXAMPLES

The present invention will be described in detail in the following with reference to the examples, while the technical scope of the present invention will not be limited to these examples.

(Materials and Methods)

[Construction siRNA Expression Lentiviral Vector for BRAF]

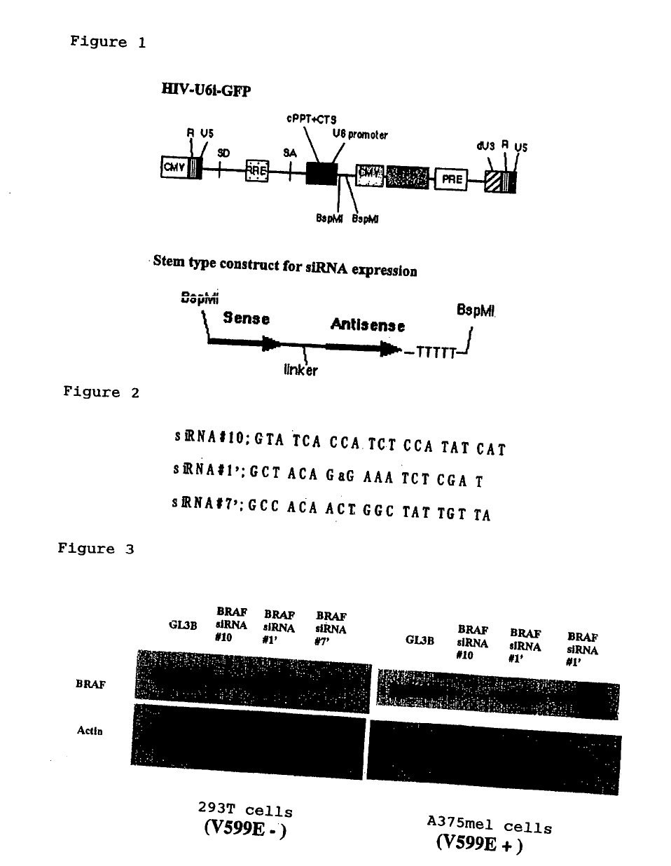

In the coding region of BRAF mRNA, RNAi target site of 19-21 mer were selected at several places at the site containing V599E mutation and at two other sites. As for the selection standard, the sequences wherein 4 or more 4 T or A are in a low are avoided, and a site wherein GC content is approximately 30-60% and initiating with AAG/A is selected, and among these, the sequence having an open structure on the secondary structure forecast program of RNA (Mfold; http://www.bioinfo.rpi.edu/appplications/mfold/old/rna/) was made as candidate. 12 types of siRNA expression lentiviral vector corresponding to the target sequence were prepared in total (7 types of regions containing V599E, 5 types of other regions). The structure of siRNA expression HIV lentiviral vector plasmid is shown in FIG. 1. cDNA of 19-21 mer, homologous to the target mRNA sequence (sense strand), linker sequence, cDNA complementary to the sense strand (anti-sense strand) and synthetic nucleotide consisting of transcription stop signal TTTTT were inserted into the BspMI site downstream of U6 promoter, and a unit expressing sense strand-linker-anti-sense strand RNA, homologous to target mRNA sequence from U6 promoter was generated. The RNA forms a loop structure in the linker part after being transcribed in the cells, and forms a stem structure between the sense strand and the anti-sense strand, it is predicted to become siRNA after being excised from the linker part in the cytoplasm by Dicer. These siRNA expressing plasmids are transfected with the other 3 types of HIV packaging plasmids (pMD.G, pRSV-Rev, pMDLg/p, RRE) to 293T cells. 48-72 hours later, lentiviral vectors generating on the culture supernatant are collected and concentrated by using Centriprep YM-50 (Millipore). The viral titer was calculated by measuring the fluorescence of GFP protein, which was also coded by the HIV vector, by flow cytometry after infecting 293T cells with the HIV vectors. Furthermore, as control virus, siRNA expression vector for firefly luciferase was produced and used.

[Examination of BRAF RNAi Effect]

After 293T cells or various malignant melanoma cell lines were infected with the siRNA expression lentiviral vectors produced as mentioned above, it was confirmed that GFP expression of each cell line was equivalent with that infected with the control vector. Then the protein was extracted, and the inhibitory effect of BRAF expression was confirmed by Western Blot Analysis. The protein extraction was performed by using 20 mM Tris-HCl (pH 7.5), 12.5 mM □-glycerophosphate, 2 mM EGTA, 10 mM NaF, 1 mM benzamide, 1% NP-40/Complete, EDTA-free (Roche), 1 mM Na₃VO₄, the supernatant was collected after cytolysis, and the protein concentration was determined by DC Protein assay kit (Promega). Moreover, as a control for the protein amount, Western Blot Analysis of actin protein was performed.

[Examination of in Vitro Growth Inhibitory Effect]

50 thousands of each of 10 types of malignant melanoma cell lines (Skmel123, 1363 mel, A375 mel, 397 mel, 501 Amel, 526 mel, 624 mel, 928 mel, 1362 mel, 888 mel) were infected with siRNA expression lentiviral vectors at 50-100 MOI (multiplicity of infection). The cell numbers were counted every 3 days until day 6-9, and the effect of BRAF RNAi on the cell growth was examined.

[Examination of the Influence to MAP Kinase Pathway]

Western Blot Analysis of ERK1 and phospholyrated ERK was performed with the protein extracted from malignant melanoma cell lines infected with siRNA expression lentiviral vector as above mentioned, and the effect of the BRAF RNAi on the activation status of MAP kinase pathway was examined.

[Animal Experiment]

Six-week old male NOD/SCID mice (Japan Clea) were subcutaneously implanted with 5×10⁶ A375 mel cells, infected with siRNA expression lentiviral vector. After implantation, the tumor volume (largest diameter×perpendicular diameter×height) was measured every 2 or 3 days until day 24. The animal experimental protocol was approved by the Laboratory Animal Care and Use Committee at Keio University School of Medicine. Mice were treated according to the Guidelines for the Care and Use of Laboratory Animals of Keio University.

(Results)

[RNAi Effect by siRNA Expression Lentiviral Vector for BRAF]

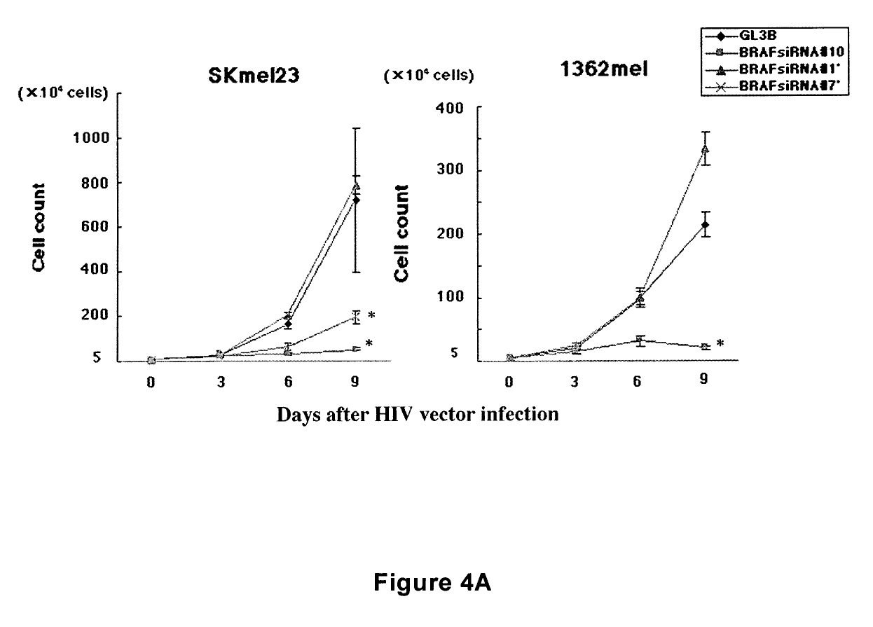

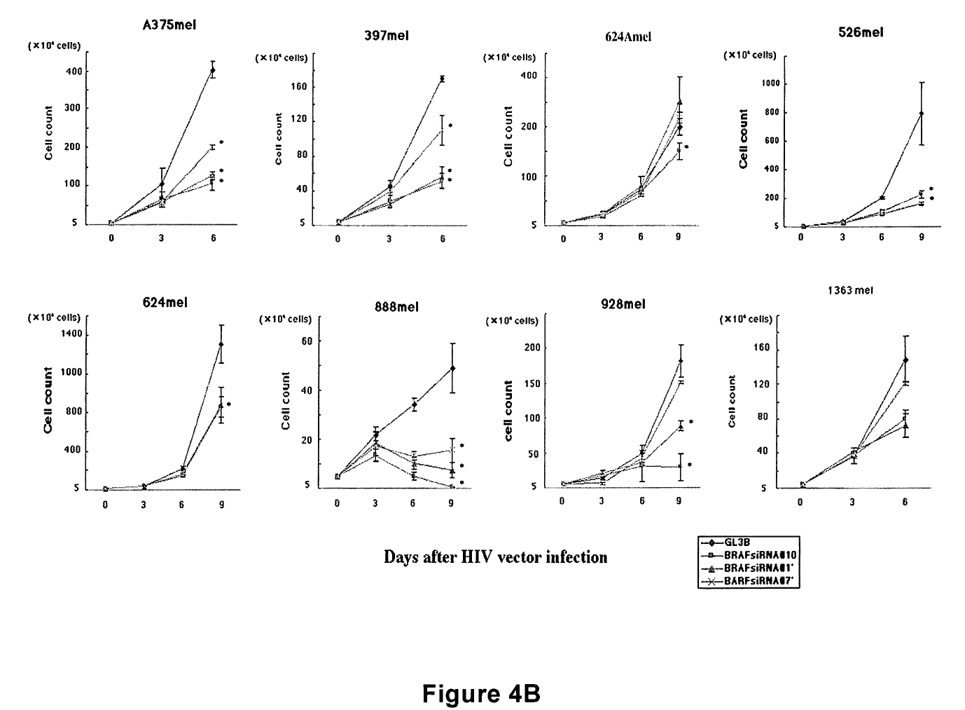

With regard to the RNAi effect, as siRNA is highly dependent of target sequence, the effect of several types of siRNA was first examined. Among the 12 types of lentiviral vector, the inhibitory effect of BRAF expression was observed with 3 types of siRNAs (#10, #1′, #7′) by Western Blot Analysis. The DNA sequences corresponding to each siRNA are shown in FIG. 2. #1′(Seq. ID No. 2) contains V599E mutation site, and targets the sequence wherein the 8th base is mutated from T to A. #10 (Seq. ID No. 4) and #7′ (Seq. ID No. 3) target the sequences which do not contain any mutation sites. The results of Western Blot Analysis of BRAF after infecting 293T cells (without V599E mutation) and A375 mel cells (with V599E mutation) with these three siRNA vectors are shown in FIG. 3. #1′ has no inhibitory effect on 293T cells that do not express the mutated BRAF V599E, however it has a significant inhibitory effect on A375 mel cells with the mutated BRAF V599E, suggesting that it inhibits specifically the expression of the mutated BRAF V599E. On the other hand, as for siRNA#10 and #7′ targeting at the wild type BRAF mRNA, a strong inhibitory effect on BRAF protein was observed in both 293T cells and A 375 mel cells, regardless of the presence or absence of V599E mutation.

[In Vitro Cell Growth Inhibitory Effect by siRNA Expressing Lentiviral Vector for BRAF]

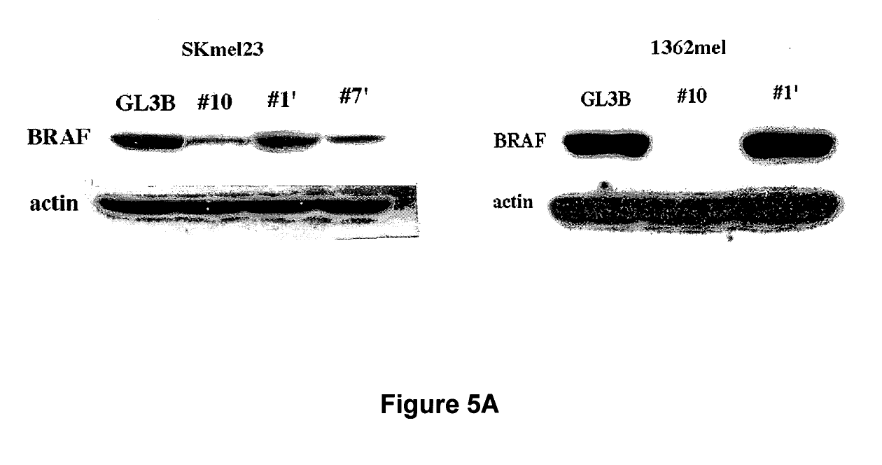

10 types of malignant melanoma cell lines were infected with 3 types of siRNA expression vectors wherein inhibitory effect of BRAF expression was observed as above mentioned at 50-100 MOI, and the effect on the cell growth was compared with the cells infected with GL3B (siRNA for firefly luciferase) expression vector as controls. The results are shown in FIG. 4. Only 2 lines, i.e. Skmel23 cells and 1362 mel cells, were negative for BRAF V599E mutation, while the other 8 lines were positive. Among all of the cell lines, siRNA#10 showed the most significant growth inhibitory effect. On the other hand, siRNA #1′ did not show growth inhibition at all for Skmel23 and 1362 mel cells without the V599E mutation, however, it showed a clear growth inhibitory effect to 5 out of 8 cell lines with the V59E mutation, and especially, the effects of siRNA#1′on 526 mel cells and 624 mel cells were equivalent or more than those with #10. Furthermore, strong cell death was induced in 888 mel cells following BRAF RANi.

[The Effect of BRAF Suppression on MAP Kinase Pathway]

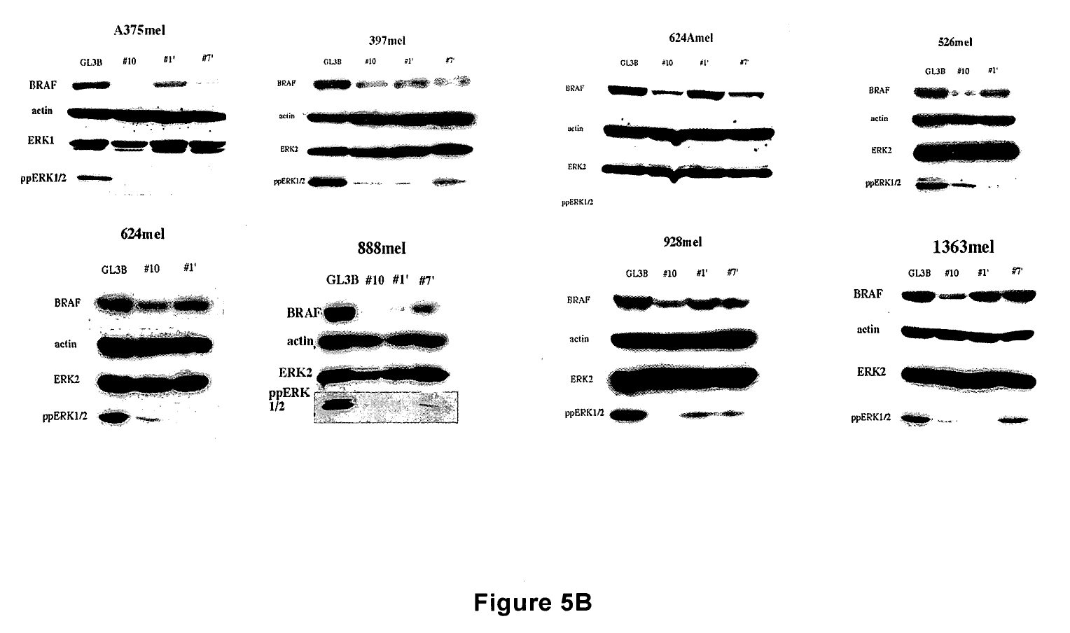

In FIG. 5, the results of the Western Blot Analysis of proteins from various malignant melanoma cell lines infected with siRNA expression vectors are shown. The suppression of BRAF protein was correlated with the decrease of the phospholyrated ERK. As no difference of ERK protein level between control GL3B and BRAF siRNAs was found, the decrease of phospholyrated ERK was not due to the decrease of ERK protein level, but to the decrease of the phospholyration, suggesting that MAP kinase pathway is inhibited.

[Inhibitory Effect of Invasion Ability of Malignant Melanoma Cell Lines]

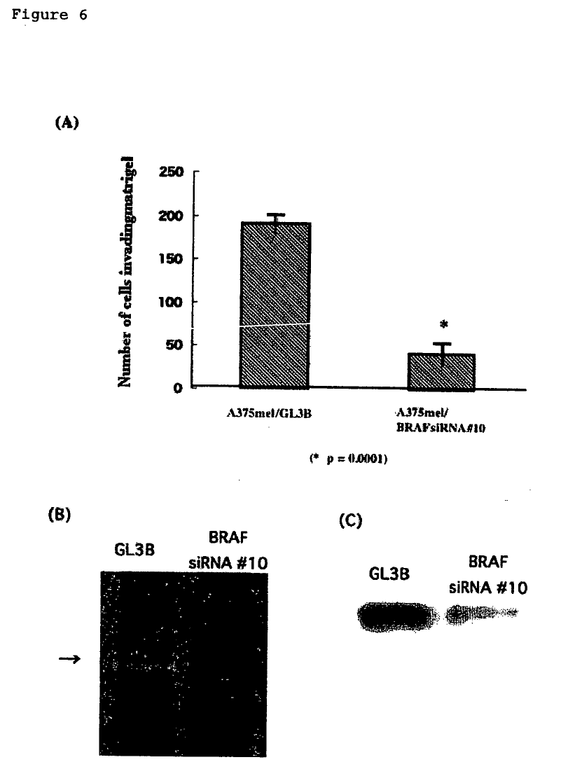

By infecting A375 mel cells with an siRNA#10 lentiviral, a significant inhibition of the invasion ability was observed by matrigel invasion assay (FIG. 6a). This was accompanied by the decrease of activation of matrix metalloproteinase-2 (FIG. 6b) and decrease of expression of □1 integrin (FIG. 6c). As the expression of these molecules, which are related to the degradation or adhesion of extracellular substrate, is controlled by MAP kinase signal, it can be suggested that the inhibition of MAPK pathway by BRAF RNAi has resulted in the inhibition of the invasion ability through the decrease of the expression of these molecules. This suggests the possibility that the inhibition of BRAF-MAPK pathway is related not only to the inhibition of cell growth but also to the inhibition of invasion ability. Therefore, the usefulness of the present invention for cancer therapy can be suggested.

[The Effect of BRAF siRNAs on the in Vivo Growth of Malignant Melanoma Cells]

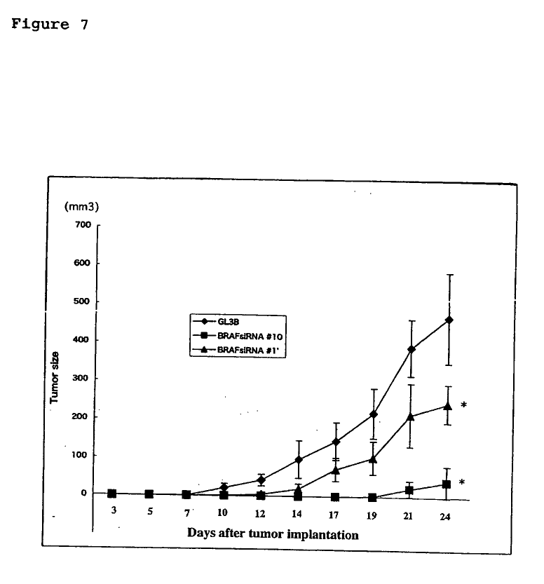

A375 mel cells infected with the siRNA#1′ lentiviral vector targeting at mutated BRAF (V599E) mRNA, or the siRNA#10 lentiviral vector targeting at wild type mRNA were implanted subcutaneously into NOD/SCID mice. After the implantation, the tumor volume was measured chronologically (FIG. 7). As shown in FIG. 7, the growth was significantly inhibited in the groups with siRNAs for BRAF, compared to that in the control group with siRNA for GL3B. Although any growth signals other than BRAF-MAPK pathway might be activated in vivo, a significant growth inhibition was observed in A375 mel cells by BRAF RNAi, suggesting that the BRAF-MAPK pathway was the major growth signal pathway of melanoma in vivo, and also the usefulness of the present invention for the treatment of cancer.

1. A double-stranded RNA that can inhibits BRAF gene expression, being consisted of a sense strand RNA and an anti-sense strand RNA being homologous to a particular sequence of BRAF mRNA.

2. The double-stranded RNA according to claim 1, wherein the BRAF gene is a mutated BRAF gene.

3. The double-stranded RNA according to claim 2, wherein the mutated BRAF gene is a mutated BRAF (V599E) gene.

4. The double-stranded RNA according to any of claims 1 to 3, wherein the particular sequence being the target of BRAF mRNA is a target sequence containing the mutation site of mutated BRAF mRNA.

5. The double-stranded RNA according to claim 4, wherein the target sequence including the mutation site of mutated BRAF mRNA consists of the RNA derived from the base sequence shown in Seq. ID No.2 and its complementary sequence.

6. The double-stranded RNA according to any of claims 1 to 3, wherein the particular sequence being the target of BRAF mRNA consists of the RNA derived from the base sequence shown in Seq. ID No. 3 or 4 and its complementary sequence.

7. The double-stranded RNA according to any of claims 1 to 6, wherein the particular sequence being the target of BRAF mRNA is the base sequence of 19-21 bp.

8. A double-stranded RNA expression cassette that can express the double-stranded RNA according to any of claims 1 to 7, being consisted of a sense strand DNA-linker-anti-sense strand DNA of the particular sequence of BRAF gene.

9. The double-stranded RNA expression cassette according to claim 8, being consisted of the base sequence shown in Seq. ID No. 5.

10. The double-stranded RNA expression cassette according to claim 8, being consisted of the base sequence shown in Seq. ID No. 6 or 7.

11. A double-stranded RNA expression vector wherein the double-stranded RNA expression cassette according to any of claims 8 to 10 is connected to the downstream of the promoter.

12. The double-stranded RNA expression vector according to claim 11, being a HIV lentiviral vector.

13. An inhibitor of BRAF gene expression, having as active ingredient the double-stranded RNA according to any of claims 1 to 7, the double-stranded RNA expression cassette according to any of claims 8 to 10, or the double-stranded RNA expression vector according to claim 11 or 12.

14. A preventive and/or therapeutic agent of cancer, having as active ingredient the double-stranded RNA according to any of claims 1 to 7, the double-stranded RNA expression cassette according to any of claims 8 to 10, or the double-stranded RNA expression vector according to claim 11 or 12.

15. The preventive and/or therapeutic agent of cancer according to claim 14, wherein the cancer is a cancer caused by the mutation or enhancement of expression of BRAF gene.

16. The preventive and/or therapeutic agent of cancer according to claim 14, wherein the cancer is a malignant melanoma.

17. A method for inhibiting BRAF gene expression, wherein the double-stranded RNA according to any of claims 1 to 7, the double-stranded RNA expression cassette according to any of claims 8 to 10, or the double-stranded RNA expression vector according to claim 11 or 12 is introduced in vivo, into tissues or cells.

18. A preventive and/or therapeutic method of cancer, wherein the double-stranded RNA according to any of claims 1 to 7, the double-stranded RNA expression cassette according to any of claims 8 to 10, or the double-stranded RNA expression vector according to claim 11 or 12 is introduced in vivo, into tissues or cells.

19. The preventive and/or therapeutic method of cancer according to claim 18, wherein the cancer is a cancer caused by the mutation or enhancement of expression of BRAF gene.

20. The preventive and/or therapeutic method of cancer according to claim 18, wherein the cancer is a malignant melanoma.


SEQUENCE LISTING (7 sequences)

SEQ ID NO: 1 - DNA, 2510 bp, Homo sapiens
cgcctcccggccccctccccgcccgacagcggccgctcgggccccggctctcggttataagatggcggcgctgagcggtggcggtggtggcggcgcggagccgggccaggctctgttcaacggggacatggagcccgaggccggcgccggccggcccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagagaaatctcgatggagtgggtcccatcagtttgaacagttgtctggatccattttgtggatggcaccagaagtcatcagaatgcaagataaaaatccatacagctttcagtcagatgtatatgcatttgggattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtggaaaaaaaaaaaaaaaaaaaaaaaaaaaaaaaaaaaaaaaccc

SEQ ID NO: 2 - DNA, 19 bp, Artificial
  siRNA#1′
gctacagagaaatctcgat

SEQ ID NO: 3 - DNA, 20 bp, Artificial
  siRNA#7′
gccacaactggctattgtta

SEQ ID NO: 4 - DNA, 21 bp, Artificial
  siDNA#10
gtatcaccatctccatatcat

SEQ ID NO: 5 - DNA, 52 bp, Artificial
  1′SENSE-LIKE-ANTISENSE-TTTTT
gctacagagaaatctcgatttcaagagaatcgagatttctctgtagcttttt

SEQ ID NO: 6 - DNA, 54 bp, Artificial
  7′SENSE-LINKER-ANTISENSE-TTTTT
gccacaactggctattgttattcaagagataacaatagccagttgtggcttttt

SEQ ID NO: 7 - DNA, 56 bp, Artificial
  10SENSE-LINKER-ANTISENSE-TTTTT
gtatcaccatctccatatcatttcaagagaatgatatggagatggtgatacttttt